(19) United States
(12) Patent Application Publication
(10) Pub. No.: US 2017/0014269 A1
(43) Pub. Date: Jan. 19, 2017

(54) SURGICAL INSTRUMENT

(71) Applicant: GEUDER AG, Heidelberg (DE)

(72) Inventors: Rene Draheim, Sandhausen (DE); Dieter Frauenfeld, Heidelberg (DE); Volker Geuder, Heidelberg (DE)

(21) Appl. No.: 15/124,751

(22) PCT Filed: Mar. 11, 2015
(86) PCT No.: PCT/DE2015/200144

§ 371 Date: Sep. 9, 2016

(30) Foreign Application Priority Data
Mar. 11, 2014 (DE) 10 2014 204 480.4
Nov. 14, 2014 (DE) 10 2014 223 304.6

(51) Int. Cl. A61F 9/007; A61B 90/30

(52) U.S. Cl. CPC A61F 9/00763; A61B 90/30; A61B 2090/306

(57) ABSTRACT

The invention relates to a surgical instrument, in particular a vitrector, comprising an outer tube and an inner tube. Both tubes functionally interact. Said surgical instrument is characterized in that the outer tube and/or the inner tube is made from a light conducting material or is coated with said type of material or said type of material comprises a light conducting fiber, core or similar, and is used to conduct light from the proximal end to the or into the area of the distal end.

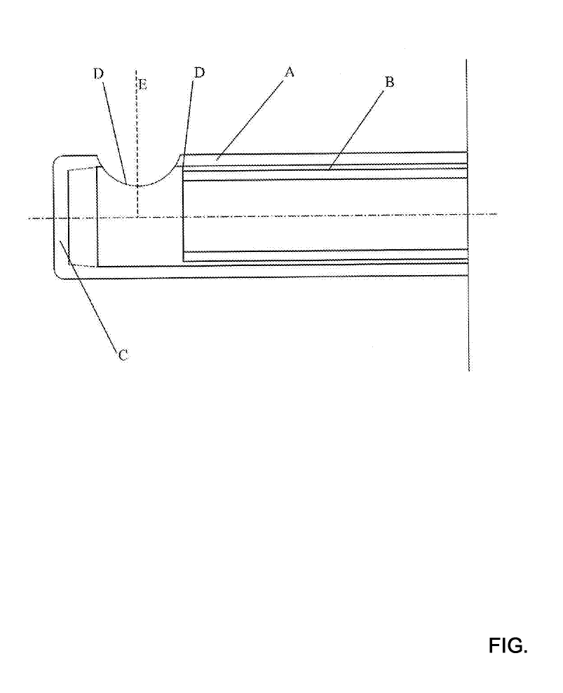

### CROSS REFERENCE TO RELATED APPLICATIONS

This application is a national stage application, filed under 35 U.S.C. §371, of International Application No. PCT/DE2015/200144, filed Mar. 11, 2015, which claims priority to German Application No. 10 2014 223 304.6, filed Nov. 14, 2014 and German Application No. 10 2014 204 480.4, filed Mar. 11, 2014, the contents of all of which as are hereby incorporated by reference in their entirety.

### BACKGROUND

1. Technical Field

The invention relates to a surgical instrument, in particular, a vitrector, comprising an outer tube and an inner tube, with the two tubes functionally interacting.

The instrument under discussion is basically a surgical instrument, in particular, a cutting instrument for removing tissue. The instrument can be used to cut the tissue on or, more specifically, in the body and to aspirate said cut tissue from or, more specifically, out of the body. In the specific case the instrument may be a very specific cutting instrument, which can be used during a vitrectomy to destroy or, more specifically, to break up the vitreous humor in the eye and to remove it from the eye. In addition, it is possible by means of this instrument to remove blood, clotted blood, connective tissue-like changes, as well as portions of the retina, during a retinal peeling.

2. Description of Related Art

With respect to the prior art reference is made, only as an example, to the patent U.S. Pat. No. 5,630,827, from which a cutting instrument that is suitable for vitreous humor operations is known. The cutting device is open on the proximal end. On the distal end said cutting device has a closed outer sheath, which is provided with a plurality of ports in the vicinity of the closed end. Each of the ports forms a slit and allows the vitreous humor tissue to penetrate into the lumen of the outer sheath. In addition, the cutting instrument has an inner sheath, which is arranged concentrically to the outer sheath and which is accommodated in the outer sheath in a manner allowing longitudinal movement, and which has a sharp edge on its distal end for the purpose of cutting the vitreous humor tissue when the inner sheath is slid forward.

The essential feature of the device, which is known from the patent U.S. Pat. No. 5,630,827, is that the inner tube has an open circumferential edge on its free frontal end and that this edge is used as the cutting edge. For this purpose this edge is shaped or, more specifically, ground. The outer edge region has a cutting effect, so that from this point on, the focus is on an outer cutting edge of the inner tube. This outer cutting edge acts together with one or more inner cutting edges of the outer tube in such a way that the tissue that has penetrated into the device is cut or, more specifically, severed when the cutting edges slide past each other. Then the tissue can be aspirated out of the interior of the device or, stated more precisely, can be aspirated through the inner tube.

A generic cutting instrument is also known from the patent WO 98/52502 for removing vitreous humor tissue. A closed outer sheath is provided on the distal end of the instrument; and in the vicinity of the distal end this outer sheath has a plurality of slits, through which the vitreous humor tissue can enter. Concentrically to the outer sheath there is a longitudinally moveable inner sheath, which has a sharp edge on its distal end for cutting the vitreous humor tissue that has penetrated into the tube. In addition, a drive mechanism is provided for the inner sheath, so that this drive mechanism can be used to move the inner sheath with its cutting edge in the region of the distal end past the slits that are arranged in the outer sheath in such a way that a cutting process takes place at the respective location.

However, in practice the known devices of the generic type are problematic in so far as it is usually necessary to provide the surgical site with light by means of a separate device, in particular, during an operation in the human eye.

### BRIEF SUMMARY

The above object is achieved by means of the features disclosed in the accompanying claims, in particular, by the fact that the outer tube and/or the inner tube is/are made of a light conductive material or is/are coated with said type of material or comprises/comprise said type of material as a light conductive fiber, core or the like and is/are used to conduct light from the proximal end to or into the area of the distal end.

It has been recognized in accordance with the invention that the surgical instrument can also be used in an ideal manner to supply light, in particular, when the outer tube and/or the inner tube is/are made of a light conductive material. Similarly it is conceivable that the outer tube and/or the inner tube is/are coated with a light conductive material. According to a third variant, it is conceivable that the material of the outer tube and/or the inner tube comprises a light conductive fiber or core, through which the light is conducted.

In any event the outer tube and/or the inner tube is/are used to conduct light from the proximal end, where the light is coupled into the outer tube and/or the inner tube, to the distal end, but at least into the area of the distal end, where the light can then emerge for purposes of illuminating the surgical site.

The light conductive material may be glass, polymethylmethacrylate, polycarbonate, etc. In this respect all light conductive materials are suitable, in any case, if they have the requisite hardness, so that the outer tube and/or the inner tube has/have cutting properties, at least in the area of a recess. An appropriate hardness is required.

As already stated above, the instrument under discussion here may be a vitrector comprising an outer cutting tube and an inner cutting tube that extends in the axial direction in said outer cutting tube. The outer cutting tube has at least one port, which forms a cutting edge. At least the front end of the inner cutting tube interacts with the cutting edge of the outer cutting tube, so that a kind of scissor function is achieved.

In the area of the cutting edge of the outer tube or at the front end of the inner tube the light output can be defined, so that an illumination of the surgical site takes place in situ.

In the case of the vitrector under discussion here the distal end of the outer cutting tube can be closed. Even at this location a defined light output is possible. In a particularly advantageous manner a glass body, preferably in the form of a lens, can be designed on the distal end for purposes of a defined light output.

It is also conceivable that the body, which is made of glass or any other light conductive material, i.e., the outer tube and/or inner tube, is coated with a material that prevents the light output and that is also used to stabilize or, more specifically, to strengthen the outer tube and/or the inner tube. At defined points, where, in particular, the light output is desired, no coating is correspondingly provided; and, therefore, the light can emerge as a function of the diffraction and refraction.

### BRIEF DESCRIPTION OF THE FIGURES

At this point there are a variety of options for designing and further developing the teaching of the present invention in an advantageous way. For this purpose reference is hereby made, on the one hand, to the patent claims, and, on the other hand, to the following explanation of a preferred exemplary embodiment of the invention with reference to the drawing. In the context of the explanation of the preferred exemplary embodiment of the invention with reference to the drawing, other generally preferred embodiments and further developments of the teaching are explained as well. In the drawing the

single FIGURE shows in a schematic view, partially, an exemplary embodiment of an inventive instrument using the example of a vitrector.

### DETAILED DESCRIPTION OF VARIOUS EMBODIMENTS

The single FIGURE shows a surgical instrument using the example of a vitrector. An outer cutting tube A and an inner cutting tube B are provided, where in this case the outer cutting tube A and/or the inner cutting tube B may be made of glass or any other light conductive material. As an alternative, it is conceivable that the outer cutting tube and/or the inner cutting tube is/are provided with a light conductive coating. In this case the cutting tubes are conventional cutting tubes, which are equipped with a light conductive coating.

The outer cutting tube A and the inner cutting tube B act together in the area of a port E, which is provided in the outer cutting tube A. That is, said outer and inner cutting tubes act across the sharp cutting edges D of said port. The cutting edges D can be defined at different clearance angles.

If the outer cutting tube A and/or the inner cutting tube B is/are made altogether of a light conductive material, then it is also advantageous if the cutting tubes A, B are coated with a light opaque material, in order to avoid an undesired light output. A light output can be provided in the area of the cutting port E in a defined manner, i.e., in the area of the cutting edges D.

It is also conceivable that the light conductive material is introduced in special grooves, is cast, sprayed or is produced as usual by coating. In any case the essential feature is that a defined light output is provided in the distal end of the outer cutting tube A, for example, in the area of the port E.

Furthermore, it is conceivable that the light output in the area of an end-sided lens at varying definition of the light output, i.e., light cone, spot, wide angle, etc., takes place at the distal end through a lens C.

If the outer cutting tube A and/or the inner cutting tube B is/are made altogether of a light conductive material, then a coating makes sure that the light output takes place only on the distal end C or in the area of the cutting port E. The coating may have the additional task of stabilizing the glass, for example, to make it also stretchable or flexible to a certain extent.

The above objects and additional advantages are achieved at least by the fact that the outer tube and/or the inner tube is/are made of a light conductive material or is/are coated with said type of material or comprises/comprise said type of material as a light conductive fiber, core or the like and is/are used to conduct light from the proximal end to or into the area of the distal end.

It has been recognized in accordance with the invention that the surgical instrument can also be used in an ideal manner to supply light, in particular, when the outer tube and/or the inner tube is/are made of a light conductive material. Similarly it is conceivable that the outer tube and/or the inner tube is/are coated with a light conductive material. According to a third variant, it is conceivable that the material of the outer tube and/or the inner tube comprises a light conductive fiber or core, through which the light is conducted.

In any event the outer tube and/or the inner tube is/are used to conduct light from the proximal end, where the light is coupled into the outer tube and/or the inner tube, to the distal end, but at least into the area of the distal end, where the light can then emerge for purposes of illuminating the surgical site.

The light conductive material may be glass, polymethylmethacrylate, polycarbonate, etc. In this respect all light conductive materials are suitable, in any case, if they have the requisite hardness, so that the outer tube and/or the inner tube has/have cutting properties, at least in the area of a recess. An appropriate hardness is required.

As already stated above, the instrument under discussion here may be a vitrector comprising an outer cutting tube and an inner cutting tube that extends in the axial direction in said outer cutting tube. The outer cutting tube has at least one port, which forms a cutting edge. At least the front end of the inner cutting tube interacts with the cutting edge of the outer cutting tube, so that a kind of scissor function is achieved.

In the area of the cutting edge of the outer tube or at the front end of the inner tube the light output can be defined, so that an illumination of the surgical site takes place in situ.

In the case of the vitrector under discussion here the distal end of the outer cutting tube can be closed. Even at this location a defined light output is possible. In a particularly advantageous manner a glass body, preferably in the form of a lens, can be designed on the distal end for purposes of a defined light output.

It is also conceivable that the body, which is made of glass or any other light conductive material, i.e., the outer tube and/or inner tube, is coated with a material that prevents the light output and that is also used to stabilize or, more specifically, to strengthen the outer tube and/or the inner tube. At defined points, where, in particular, the light output is desired, no coating is correspondingly provided; and, therefore, the light can emerge as a function of the diffraction and refraction.

Finally it must be explicitly pointed out that the exemplary embodiment of the inventive surgical instrument described above is used only to explain the claimed teaching, but the teaching is not restricted to the exemplary embodiment.

1-6. (canceled)

7. A surgical instrument comprising:
an outer tube; and
an inner tube functionally interacting with the outer tube,
wherein:
at least one of the outer tube or the inner tube is either made of a light conductive material, is coated with a light conductive material, or comprises a light conductive fiber core; and
the light conductive material is configured to conduct light from a proximal end at least to an area of a distal end of the surgical instrument.

8. The surgical instrument of claim 7, wherein the instrument is a vitrector.

9. The surgical instrument of claim 7, wherein the light conductive material is at least one of glass, PMMA, or polycarbonate.

10. The surgical instrument of claim 7, wherein:
the instrument is a vitrector comprising an outer cutting tube and an inner cutting tube that can be moved axially therein;
at least one port of the vitrector forms a cutting edge; and
a light output is defined in an area of the cutting edge.

11. The surgical instrument of claim 10, wherein:
the outer cutting tube is closed on the distal end; and
the distal end further defines the light output.

12. The surgical instrument of claim 7, wherein a glass body is formed on the distal end for purposes of a defined light output.

13. The surgical instrument of claim 12, wherein the glass body is a lens.

14. The surgical instrument of claim 7, wherein the light conductive material of at least one of the outer tube or the inner tube is coated in order to prevent an undesired light output and uncoated only at defined light output points.
